(19) United States
(12) Patent Application Publication
(10) Pub. No.: US 2023/0330019 A1
(43) Pub. Date: Oct. 19, 2023

(54) EMULSION FORMULATIONS OF POORLY WATER-SOLUBLE COMPOUNDS

(71) Applicant: Mark Michael MENNING, San Rafael, CA (US)

(72) Inventor: Mark Michael MENNING, San Rafael, CA (US)

(21) Appl. No.: 18/134,896

(22) Filed: Apr. 14, 2023

Related U.S. Application Data
(60) Provisional application No. 63/331,628, filed on Apr. 15, 2022.

(51) Int. Cl. A61K 9/107; A61K 47/10; A61K 31/05; A61K 31/352; A61K 36/28; A61K 31/60; A61K 31/7048; A61K 9/00; A61K 31/12; A61K 36/9066; A61K 31/506; A61P 17/14

(52) U.S. Cl. CPC A61K 9/107; A61K 47/10; A61K 31/05; A61K 31/352; A61K 36/28; A61K 31/60; A61K 31/7048; A61K 9/0014; A61K 31/12; A61K 36/9066; A61K 31/506; A61P 17/14

(57) ABSTRACT

An aqueous emulsion system substantially or completely devoid of an oil phase. The aqueous emulsion can be used as a platform to solubilize poorly water-soluble chemical compounds for use in a variety of fields, including pharmaceutical, cosmetic, skin care, personal care, food, and industrial arenas. The aqueous emulsion can be delivered to a human or animal via a variety of modes to treat a diverse number of ailments, such as a topical composition to promote hair growth or treat skin disorders. Stable and transparent formulations of the aqueous nano-emulsions described herein can be used to treat and/or prevent hair loss, to treat warts and cold sores, or to treat varicose veins.

### RELATED APPLICATIONS

This application claims priority under 35 U.S.C. § 119(e) to U.S. Provisional Patent Application No. 63/331,628 filed Apr. 15, 2022, which is incorporated herein by reference.

### BACKGROUND OF THE INVENTION

Emulsions are used in a wide variety of industrial, cosmetic, personal care, skin care and pharmaceutical products including ocular, topical, mucosal, intravenous, intramuscular, sublingual and oral products. Emulsions are also employed as precursors to prepare polymer microparticles, solid lipid nano-particles, inorganic nano-particles and oil-filled micro-capsules, and have been developed as precursors to magnetic particles for imaging, diagnostics, and drug delivery. Even with their widespread use, they remain an underutilized drug product format.

It can be challenging to combine a poorly water-soluble chemical compound in a water-based system. Formulators in the biological, chemical, pharmaceutical, cosmetic, and other industries have developed several methods to overcome this challenge. One of the more studied methods is to form an emulsion colloid by mixing an oil phase with a water phase in combination with an emulsifying agent. This type of emulsion colloid has an oil phase, a water phase, and a surfactant or other emulsifier, and may also contain a co-solvent to aid in solubilizing the poorly water-soluble chemical compound.

Oil and water are immiscible liquids that do not form a homogeneous mixture when added together. However, when mixed with an emulsifying agent, they can form an emulsion (e.g., oil-in-water, water-in-oil, or a water-in-oil-in-water triple emulsion) in which a poorly water-soluble (but oil-soluble) chemical compound can be dispersed. An emulsion is a thermodynamically stable isotropic system in which two immiscible liquids (e.g., oil and water) are mixed to form a single dispersion by means of an emulsifying agent (e.g., surfactant+co-surfactant). One of the immiscible liquids is dispersed in the form of very small globules (internal phase) throughout the other immiscible liquid (external phase). The resulting emulsion is a kinetically stable and clear (i.e., transparent or translucent) dispersion of two immiscible phases. Even though the liquids that form them may be clear, emulsions can appear cloudy or colored because light is scattered by the suspended particles in the mixture.

A poorly water-soluble chemical compound can detrimentally affect the appearance and efficacy of the compound in the product. Active chemical compounds that are poorly water-soluble exhibit diminished bioavailability in a product (e.g., such as a pharmaceutical, cosmetic or food product), especially in products with high water concentrations. Some active compounds cannot be orally dosed because they are not effective or can cause harm (e.g., fast metabolism due to short half-life, toxicity issues, side effects, etc.). Oil-in-water and water-in-oil emulsions comprising oil, water, surfactants (or other emulsifying agents), and co-solvents have been formulated and studied with poorly water-soluble active ingredients. These studies have demonstrated a general need for having an oil phase in a composition for forming an aqueous based emulsion system containing one or more different types of poorly water-soluble active and inactive compounds.

Notwithstanding the varied kinds of formulations that have been developed, their effectiveness and side effects have been less than desirable. There is a demand for better formulations to effectively deliver poorly water-soluble active and inactive chemical compounds to a human or an animal. There remains a need for improved formulations containing poorly water-soluble active and inactive chemical compounds. There exists a need for new aqueous based topical compositions containing poorly water-soluble active and inactive chemical compounds.

The approach described in this patent seeks to meet these needs by providing a novel aqueous based emulsion system without the substantial or complete presence of an oil phase. The emulsion system described herein can be used as a platform to formulate oil-free aqueous compositions containing poorly water-soluble active and inactive compounds, which can be beneficial to treat a variety of skin disorders and other types of diseases and ailments.

### SUMMARY

Described herein is an aqueous emulsion system without the substantial or complete presence of an oil phase comprising the combination of:
(i) about 2.5% to about 50% by weight of diethylene glycol monoethyl ether (DEGEE);
(ii) about 5% to about 20% by weight of a polyethylene glycol (PEG: H(OCH₂CH₂)ₙOH) derivative of castor oil, where n is 2 to about 1,000;
(iii) about 60% to about 92% by weight of water; and
(iv) about 0.05% to about 2.0% by weight of a poorly water-soluble chemical compound that is dispersed in a substantially or completely oil-free emulsion.

The emulsion system can operate as a platform to deliver a poorly water-soluble chemical compound via a variety of ways, such as, orally, sublingually, topically, or via an infusion. It is particularly useful for topical cutaneous (skin) therapeutic applications.

In some embodiments, n is equal to about 30 to about 40 in the PEG chain of the polyethylene glycol derivative of castor oil. In certain embodiments, the polyethylene glycol derivative of castor oil is PEG 40 hydrogenated castor oil (PEG 40 HCO).

The oil-free aqueous composition can be a mini-, micro- or nano-emulsion. The nano-emulsions can have a d50 nano-particle size and a number weighted mean nano-particle size each of less than about 150 nm. Optimizing the loads of the ingredients in the composition can reduce or increase the particle size and droplet size. An exemplified embodiment has a d50 nano-particle size and a number weighted mean nano-particle size of each less than about 50 nm.

The oil-free aqueous emulsion can be advantageously used for solubilizing poorly water-soluble active and inactive ingredients. In an embodiment, the active ingredient can be a natural product, such as a flavonoid (e.g., flavanol) or a polyphenol. Exemplified below are oil-free aqueous emulsions containing one or more of the following different natural products—fisetin, trans-resveratrol, milk thistle extract comprising silymarin, quercetin (or dihydroquercetin), (−)-epigallocatechin-3-gallate (EGCG), salicylic acid, Centipeda minima, curcumin (or turmeric), rutin, diosmin, and imatinib or imatinib mesylate. These natural products have been the subject of many studies for potential uses, including the treatment and prevention of alopecia.

In one example, a hair growth composition was prepared comprising:
(i) about 2.5% to about 20% by weight of DEGEE;
(ii) about 5% to about 10% by weight of PEG 40 HCO;
(iii) about 60% to about 92% by weight of water; and
(iv) a poorly water-soluble active chemical compound comprising:
   (a) about 0.075% to about 0.250% by weight of fisetin;
   (b) about 0.075% to about 0.150% by weight of trans-resveratrol; and/or
   (c) about 0.125% to about 0.250% by weight of a milk thistle extract comprising about 0.1% to about 0.2% by weight of silymarin.

The invention further includes methods of application that can be beneficial for treating a variety of skin disorders. The examples show various topical applications. Two exemplified skin disorders that can be effectively treated with the formulations described herein are alopecia and human papillomavirus (HPV) warts by topically applying a therapeutically effective amount of a formulation with the required active ingredient for a therapeutically effective amount of time to an affected area of the skin disorder in a human or animal in need thereof. The emulsion system can be used as a platform to create formulations with a variety of poorly water-soluble chemical compounds, including natural and synthetic, active and inactive compounds. The emulsions can be employed for applications other than topical (e.g., ocular, mucosal, intravenous, intramuscular, and oral products. Depending on the active ingredient(s) that are selected, the emulsions can be used to treat a plethora of ailments (e.g., psoriasis, cancer, inflammation, and pigmentary disorders, such as vitiligo and melasma) other than are exemplified herein.

The invention further includes a method of formulating an oil-free aqueous emulsion.

The foregoing and other objects and features of the disclosure will become more apparent from the following detailed description, which proceeds with reference to the accompanying figures. Further embodiments, forms, features, aspects, benefits, objects, and advantages of the invention shall become apparent from the detailed description and figures provided herewith.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention. The features, objects and advantages will become more readily apparent when consideration is given to the detailed description below. The detailed description makes reference to the following drawings:

FIG. 1: Photographs of a male human scalp (after having had a haircut) (a) before and (b) after four weeks of treatment with a nano-emulsion formulation containing fisetin;

FIG. 2: Photographs of the left foot of a human male having a wart mass (a) before and (b) after four weeks of treatment with a nano-emulsion formulation containing (−)-epigallocatechin-3-gallate (EGCG) and quercetin;

FIG. 3: Photographs of the right foot of a human male having two wart masses (a) before and (b) after four weeks of treatment with a nano-emulsion formulation containing (−)-epigallocatechin-3-gallate (EGCG) and quercetin; and

FIG. 4: (a) Photograph showing the removal of a wart mass on a human male foot and (b) Pictorial of viruses and roots in a wart mass.

While the present invention is susceptible to various modifications and alternative forms, exemplary embodiments thereof are shown by way of example in the drawings and are herein described in detail. It should be understood, however, that the description of exemplary embodiments is not intended to limit the invention to the particular forms disclosed. On the contrary, the intention is to cover all modifications, equivalents and alternatives falling within the spirit and scope of the invention as defined by the embodiments herein and the claims below. Reference should therefore be made to the embodiments and claims for interpreting the scope of the invention.

### DETAILED DESCRIPTION

The novel oil-free aqueous emulsion system described herein can be used as a platform to solubilize poorly water-soluble active and inactive ingredients. Below are definitions for some of the terms used in this disclosure.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, the phrase can mean one, two, three, four, five, six, ten, 100, or any upper limit approximately 10, 100, or 1000 times higher than a recited lower limit.

As will be understood by the skilled artisan, all numbers, including those expressing quantities, properties, purities, reaction conditions, and so forth, are approximations and are understood as being optionally modified in all instances by the term “about.” These values can vary depending upon the desired goals sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by the skilled artisan, all numbers, including those expressing quantities, properties, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language, such as “up to”, “at least”, “greater than”, “less than”, “more than”, “or more”, and the like, include the number recited, and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The terms “contacting” or “applying” refer to the act of touching, making contact, or of bringing to immediate or close proximity, including at the cellular or molecular level, for example, to bring about a physiological reaction, a chemical reaction, or a physical change, e.g., on the skin of a human or animal, in an emulsion, in a solution, in a reaction mixture, in vitro, or in vivo.

An “effective amount” refers to an amount effective to bring about a recited effect, such as, an amount of an ingredient or a combination of ingredients necessary to form a product(s) in a reaction mixture, an amount of a composition necessary to bring about a therapeutic effect, or an amount of time necessary to apply a composition to bring about a therapeutic effect. Determination of an effective amount is typically within the capacity of persons skilled in the art, especially in light of the detailed disclosure provided herein. Thus, an “effective amount” generally means an amount that provides a desired effect.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

A “topical” drug product is one that is administered to the cutaneous (skin) or other external body tissue surface (such as a mucous membrane) to treat a condition of the skin or other tissue to which the drug product is applied. Common formats for topical formulations include ointments, lotions, creams, solutions, gels, and foams.

“Dispersion” in chemistry is a mixture in which fine particles of one substance are scattered throughout another substance. The two phases may be in the same or different states of matter (e.g., gas, fluid, liquid, or solid). A dispersion can be classified as a suspension, colloid, or solution. Generally, the particles in a solution are of molecular or ionic size; those in a colloid are larger, but too small to be observed with an ordinary microscope; and those in a suspension can be observed under a microscope or with the naked eye. A coarse mixture (e.g., sand mixed with sugar) is usually not thought of as a dispersion.

The dispersion of fine particles in a liquid plays an important role in many processes of particle technology. Particles that are suspended in a liquid phase are referred to as solid/liquid dispersions. There are two general ways to produce a solid/liquid dispersion: (1) size reduction of the solid phase in a liquid to ensure uniformity, and (2) precipitation of fine particles in a liquid phase. Surfactants may be used to enhance the wettability of the solids in the liquid phase. Viscosity modifiers may be incorporated in the liquid phase to reduce sedimentation of the particles dispersed in the liquid phase.

As used herein, “precipitation” is the sedimentation of a solid material, a precipitate, from a liquid solution in which the material is present in amounts greater than its solubility in the liquid.

An “emulsion” can be defined as a colloid consisting of two or more non-homogenous type of liquids wherein one of the liquids contains the dispersion of the other liquid. Emulsions basically consist of a dispersion of two liquids that are immiscible with each other. One of the liquids acts as the dispersion medium and the other acts as the dispersed phase. In other words, emulsions are colloids in which both the dispersed phase and dispersion medium are liquids. In an oil-in-water emulsion, the oil forms droplets that disperse throughout the water. The term emulsion can also be applied to a group of mixed systems, such as solutions, gels or suspensions.

The International Union of Pure and Applied Chemistry (IUPAC) defines an emulsion as a fluid system in which liquid droplets are dispersed in a liquid. The droplets may be amorphous, liquid-crystalline, or any mixture thereof. The diameters of the droplets constituting the dispersed phase usually range from approximately 10 nm to 100 μm; i.e., the droplets may exceed the usual size limits for colloidal particles. An emulsion is termed an oil/water (o/w) emulsion if the dispersed phase is an organic material (an ‘oil’) and the continuous phase is water or an aqueous solution. It is termed a water/oil (w/o) emulsion if the dispersed phase is water or an aqueous solution and the continuous phase is an organic material (an ‘oil’).

A substantially or completely ‘oil-free’ emulsion is an emulsion that does not contain a continuous or dispersed oil phase, or contains such a small amount of an oil phase (continuous or dispersed) that it does not materially affect the properties of the emulsion.

Emulsions contain both a continuous phase and a dispersed phase, with the boundary between the phases called the “interface”. Emulsions can have a cloudy appearance due to many phase interfaces scattering light passing through the emulsions. They can appear to have white color when the light is dispersed in equal proportions. If an emulsion is dilute, then higher-frequency and low-wavelength types of light will be scattered in more fraction. For example, one kind of this type of an emulsion will appear blue in color (the “Tyndall effect”.) The term “micro-emulsion” (ME) came of widespread use in the 1950's to describe a multi-phase system consisting essentially of water, oil, surfactant and alcohol. MEs are thermodynamically stable micellar colloidal dispersions having a particle size of about 10-100 nm. MEs can form a clear or transparent solution, usually with simple mixing of the ingredients. They are low-viscosity systems containing an aqueous phase, an oil phase, a surfactant, and a co-surfactant. MEs are a type of micellular dispersion that generally require a higher concentration of surfactant loads versus typical micellular solutions.

The term “nano-emulsion” (NE) is also a clear or transparent colloidal dispersion. However, in contrast to a ME, a NE is not thermodynamically stable and frequently requires high shear processing to form.

“Micelles” are an aggregate of molecules in a colloidal solution. In aqueous systems, they are organized structures formed by the surfactants in water. Clear micellular solutions in aqueous oil-in-water dispersions generally use surfactants and/or solvents to reduce the oil droplet size to < about 50 nanometers. This point is referred to as the critical micelle concentration (CMC). Above the CMC, these structures surround hydrophobic ingredients, like fragrances and oils, and can form clear or transparent dispersions if the right surfactants and/or solvents are used at high enough concentrations.

An “emulsifier” is a substance that stabilizes an emulsion by increasing its kinetic stability. Emulsifiers are a part of a broader group of compounds known as surfactants, or “surface-active agents”. Surfactants (emulsifiers) are compounds that are typically amphiphilic, meaning they have a polar or hydrophilic (i.e., water-soluble) part and a non-polar (i.e., hydrophobic or lipophilic) part. Because of this dual modality, emulsifiers tend to have more or less solubility either in water or in oil. Emulsifiers that are more soluble in water (and conversely, less soluble in oil) will generally form oil-in-water emulsions, while emulsifiers that are more soluble in oil (and conversely, less soluble in water) will form water-in-oil emulsions.

“Miscibility” is the property of two substances to completely mix to form a homogeneous solution. Usually, the term is used to describe liquid mixtures, but can also be applied to solids and gases. Two substances are miscible if they mix in all proportions or concentrations to form a solution. In other words, it doesn't matter whether you mix them equally or one component is present in a greater amount than the other. Two substances are immiscible if they don't completely mix to form a solution. When combined, immiscible substances separate into layers or form a heterogeneous mixture. Miscibility is the capability of a mixture (for example, a mixture of two different liquids) to form a single phase over defined ranges of temperature, pressure, and composition. The existence of a single phase depends on the chemical structure, molar-mass distribution, and molecular architecture of the components present. If a mixture is thermodynamically metastable, it can de-mix if suitably nucleated. If a mixture is thermodynamically unstable, it can de-mix by spontaneous decomposition (i.e., without nucleation) or by nucleation and growth if suitably nucleated.

“Solubility” generally refers to the degree to which a substance dissolves in a solvent to make a solution (usually expressed as grams of solute per liter of solvent). Solubility of one fluid (liquid or gas) in another may be complete (totally miscible; e.g., methanol and water) or partial (e.g., oil and water dissolve only slightly). In general, “like dissolves like” (e.g., aromatic hydrocarbons dissolve in each other but not in water). Solubility is the ability of a solid, liquid, or gaseous chemical substance (referred to as the solute) to dissolve in a solvent (usually a liquid) and form a solution. The solubility of a substance depends on the types of solvent used, their polarity, and the temperature and pressure to which they are subjected. The solubility of a substance in a particular solvent is measured by the concentration of the saturated solution. A solution is considered saturated when adding additional solute no longer increases the concentration of the solution. The degree of solubility ranges widely depending on the substances, from infinitely soluble (fully miscible), such as ethanol in water, to poorly soluble, such as silver chloride in water. The term “insoluble” is often applied to poorly soluble compounds. Under certain conditions, the equilibrium solubility can be exceeded, yielding a supersaturated solution. Solubility does not depend on particle size; given enough time, even large particles will eventually dissolve.

“Poorly water-soluble chemical compounds”: The United States Pharmacopeia (USP) designates the solubility of drugs as parts of solvent required for one part solute. The solubility of a drug comprising active and/or inactive ingredients may be expressed in a number of ways, such as by weight percentage, molarity, and molality. The USP lists the solubility of a drug as the quantity of milliliters of solvent in which 1 g of solute will dissolve and assigns a descriptive term in the summary provided below.

| | Part of solvent required |
|---|---|
| USP solubility: Descriptive term | per part of solute |
| Very soluble | less than 1 |
| Freely soluble | from 1 to 10 |
| Soluble | from 10 to 30 |
| Sparingly soluble | from 30 to 100 |
| Slightly soluble | from 100 to 1000 |
| Very slightly soluble | from 1000 to 10,000 |
| Practically insoluble | 10,000 and over |

The chemical compounds employed in the examples below meet the criteria for slightly soluble, very slightly soluble, and practically insoluble, which are collectively defined as “poorly water-soluble” chemical compounds.

Solubility table of exemplified compounds as
they relate to the USP solubility definition.

| | Water Solubility | | |
|---|---|---|---|
| Compound | (mg/mL) | mass_water/mass_solute | Definition |
| Fisetin | 0.01 | 100,000 | practically insoluble |
| Quercetin | 0.002 | 500,000 | practically insoluble |
| EGCG | 5.0 | 200 | slightly insoluble |
| Curcumin | 0.00006 | 16,666,667 | practically insoluble |
| Salicylic Acid | 2.48 | 403 | slightly insoluble |
| trans-Resveratrol | 0.03 | 33,333 | practically insoluble |
| Silymarin | 0.4 | 2,500 | very slightly insoluble |
| Dihydroquercetin | 0.4 | 2,500 | very slightly insoluble |
| Rutin | 0.125 | 8,000 | very slightly insoluble |
| Disomin | 0.000019 | 52,631,579 | practically insoluble |
| Imatinib Free Base | 0.009 | 111,111 | practically insoluble |

The term “poly(ethylene glycol)” or “PEG” refers to the compound H(OCH₂CH₂)ₙOH, wherein n is 2 to about 1,000 in the PEG chain, or a derivative thereof. In various embodiments, the molecular weight of a PEG chain can be about 500 to about 200,000. In certain embodiments, the PEG group can have a molecular weight of about 500 to about 20,000; about 2,000 to about 15,000; about 3,500 to about 12,000; or about 3,000 to about 9,000. In other embodiments, the PEG groups can have a molecular weight of about 4,000 or about 7,000. The PEG group can be capped at its terminal end with a protecting group, such as an acetyl group or an alkyl group, for example, a methyl or an ethyl group. In a polymer, two or more ethylene glycol segments can form a poly(ethylene glycol) (“PEG”) chain. Typically, the number (n) will be much greater than two segments, such as about 5, about 10, about 20, about 50, about 100, about 200, about 300, about 400, about 500, about 600, or about 800 segments, or any range in between any two of the aforementioned values. In certain embodiments, n is about 30 to about 40. In an exemplified embodiment, n is 40.

The PEG chain can have a molecular weight of about 200 to about 40,000 g/mol. Some embodiments can have PEG moieties of about 300 to about 30,000 g/mol, or about 400 to about 20,000 g/mol. Some embodiments can have PEG moieties with molecular weights of about 5,000, about 6,000, about 8,000, about 10,000, about 12,000, about 15,000, about 20,000, about 25,000, or about 30,000, or any range in between any two of the aforementioned values. The PEG groups can be single chains, double chains, branched chains, or cyclic or polycyclic groups. In certain circumstances, higher molecular weight PEG chains may be useful to increase the solubility of block copolymers in conjugating multiple types of water-insoluble drugs and/or molecules.

While the description and examples are focused on topical applications of an emulsion containing a poorly water-soluble active ingredient(s), it can be readily seen that the invention can serve as a platform for formulating and delivering poorly water-soluble inactive ingredient(s), and applying an emulsion via modes other than topical, such as ocular, mucosal, intravenous, intramuscular, sublingual, and oral.

Emulsions, particularly micro-, mini-, and nano-emulsions, can be advantageous for topically delivering an active (and/or inactive) ingredient(s) into different layers of the epidermis and into systemic circulation of the body. Conventional topical treatments, such as gels and creams, generally have low efficiency, and poor cosmetic and aesthetic appeal, which can lead to poor patient compliance or adherence, while systemic and photo therapy often produce significant adverse side effects. The small particle and droplet sizes that are present in a nano-emulsion (˜5-500 nm) drug formulation can enhance the appearance, delivery and penetration of the drug through the epidermis and layers of the skin.

The art of formulating a topical composition to treat a disease, ailment or disorder through the skin entails a sophisticated application of multiple scientific disciplines, e.g., chemistry, biology, physics, mathematics, and materials. A common challenge to a formulator is how best to mix an active ingredient(s) with other (active or inactive) ingredients to provide an efficacious and easy to use composition. It has been determined through experimentation that the oil-free aqueous emulsions of the invention can be used as a platform to solubilize poorly water-soluble active (as well as inactive) ingredients. The compositions and methods of use described herein can be helpful for producing a variety of products in multi-disciplinary areas.

The topical emulsion system described herein does not (substantially or completely) possess an oil phase and can be formulated as a mini-, micro-, or nano-aqueous emulsion. It comprises:
(i) about 2.5% to about 50% by weight of diethylene glycol monoethyl ether (DEGEE);
(ii) about 5% to about 20% by weight of a polyethylene glycol (PEG: H(OCH₂CH₂)ₙOH) derivative of castor oil, where n is 2 to about 1,000;
(iii) about 60% to about 92% by weight of water; and
(iv) about 0.01% to about 2.0% by weight of a poorly water-soluble chemical compound that is dispersed in a substantially or completely oil-free topical emulsion.

In an embodiment, n equals about 30 to about 40 in the PEG chain. In certain embodiments, the PEG derivative of castor oil can be PEG 40 (n=40) hydrogenated castor oil (PEG 40 HCO).

The emulsion system can be used as a formulation platform to create a nano-emulsion of an active ingredient(s) (e.g., a flavonoid) without the substantial or complete presence of an oil phase in the emulsion. The emulsion platform can create nano-particles (and nano-droplets) that improve the appearance of a product, and efficiently deliver active (and/or inactive) ingredients topically, while addressing solubility issues inherent with poorly water-soluble chemical compounds, which adversely affects their bioavailability. The formulation platform can be used to create compositions that maintain transparency and solubility upon further dilution with water without the need for an oil phase used in conventional mini-, nano- and micro-oil-in-water or water-in-oil emulsions. The formulation platform offers a wide range of applications for topical delivery of poorly water-soluble chemical compounds that cannot be effectively dosed orally. The solubilized composition can be mixed into a base, such as a lotion or cream, to enhance permeation and delivery properties of the chemical compounds.

Formulations according to the invention provide an emulsion platform that can be useful for making products in many industries (e.g., pharmaceutical, cosmetic, personal care, industrial, food, etc.). In the pharmaceutical area, it can be used to make products that treat a variety of ailments and disorders, such as hair loss, psoriasis, cancer, inflammation, and pigmentary disorders (e.g., vitiligo and melasma).

The emulsion platform can be designed to provide prophylactic benefits (e.g., UV protection, anti-aging protection, dietary supplements for preventing and/or treating a variety of medical issues, etc.) or absorption of drugs that undergo high first-pass metabolism during oral or other modes of delivery. It can also be used to encapsulate gene therapy products, such as siRNA, cccDNA, mRNA, etc.

In embodiments, a topical composition of the invention comprising an active ingredient may further comprise an inactive ingredient, such as a co-solvent, co-emulsifier, cationic, ionic or non-ionic co-surfactant, stabilizer, thickener, anti-oxidant, conditioner, moisturizer, emollient, penetration enhancer, preservative, nutrient, fragrance, pigment, colorant, dispersion agent, anti-pruritic agent, anti-perspirant, anti-psoriatic agent, anti-seborrheic agent, anti-aging agent, anti-wrinkle agent, skin lightening agent, depigmenting agent, vitamin, nutrient, aroma, preservative, stabilizer, or a combination thereof.

The composition may comprise one or more active ingredients. For example, the active ingredient can be a natural product, such as a flavonoid (e.g., flavanol) or a polyphenol. Exemplary natural products include, but are not limited to, fisetin, trans-resveratrol, a milk thistle extract comprising silymarin, quercetin, dihydroquercetin, curcumin (and/or turmeric), (−)-epigallocatechin-3-gallate (EGCG), rutin, diosmin, an extract of Centipeda minima, imatinib, and imatinib mesylate. The active ingredient can be a hair growth promoter, or an antibiotic, antiseptic, antifungal, antibacterial, anti-inflammation, analgesic, antiviral, anesthetic, anti-cancer (e.g., imatinib or its mesylate salt) or anti-acne agent.

Optionally, the composition may comprise a ‘natural’ or ‘essential’ oil. Natural oils have been studied and marketed for a variety of skin disorders and other ailments in the pharmaceutical, food, personal care, skin care and cosmetic fields. Some examples include coconut, almond, grapeseed, olive, sunflower seed, argan, rosehip see, jojoba, marula, tea tree, and safflower oils. Similarly, essential oils have been the subject of a variety of studies in these fields. Examples of essential oils include lavender, chamomile, sandalwood, clary sage, rosemary, frankincense, geranium, neroli, lemon, lemongrass, cinnamon, tea tree, peppermint, wintergreen, eucalyptus, patchouli, pomegranate, carrot seed, tangerine, ylang ylang, rose, myrrh, and jojoba oils.

Though essential and natural oils are both derived from plants, there are differences between the two classes. Essential oils are distilled from the leaves, roots, bark, and other aromatic portions of a plant or botanical. The oils evaporate and have a concentrated aroma. Accordingly, essential oils are concentrated, hydrophobic liquids containing volatile aromatic compounds derived from plants/botanicals. Due to their high potency, essential oils can cause irritation to the skin and are usually diluted with a carrier oil to alleviate this issue.

In contrast, natural oils are usually pressed from the fatty portions of a plant or botanical, such as the seeds, nuts or kernels. Chemically, natural oils are triglycerides in which the glycerin is esterified with three fatty acids. They are the main constituent of vegetable oils and animal fats. Natural oils are generally gentler on the skin than essential oils and generally do not require the addition of a carrier oil to use them.

In embodiments, a topical composition can be in the form of an ointment, a cream, an emulsion, a lotion, a paste, an unguent, a gel, a tincture, a sunscreen a spray, or other topical dosage form. The emulsions described herein may further comprise a cleansing agent, an emollient, or an aromatic chemical compound. Ointments, creams, lotions, and unguents according to the invention may further comprise a wax, alcohol, or a petroleum-based mollifying agent.

Gels according to the invention optionally comprise a vegetable oil, for example, of up to about 5% by weight of the total composition. Gels may further comprise water and/or a thickening agent. The thickening agent may be a natural polysaccharide, such as xanthan gum, carrageen, alginate, and/or cellulose gum.

Pastes according to the invention may optionally comprise aloe gel and/or beeswax.

Lotions according to the invention may optionally comprise cetyl alcohol, an emulsifier, a fragrance, glycerol, petroleum jelly, a dye, one or more preservatives and/or a stabilizing agent.

A sunscreen composition according to the invention may optionally comprise a UV-absorbing or barrier agent in an amount, for example, of between about 0.1% and about 10% by weight of the total composition. Exemplary UV-absorbing compounds include, but are not limited to, benzone compounds, glyceryl PABA, roxadimate, octocrylene, octyl methoxycinnamate, ethoxyethyl p-methoxycinnamate, homomenthyl salicylate, ethylhexyl salicylate, trolamine salicylate, ecamsule, ensulizole, bemotrizinol, and bisoctrizole. Exemplary UV-barrier compounds include, but are not limited to, zinc oxide and titanium dioxide.

The emulsion system of the invention may comprise an antibiotic, antiseptic, antifungal, analgesic, antiviral, and/or stabilizer agent. Some examples of these agents are listed below.

Antibiotic agents include, but are not limited to, ampicillin, bacampicillin, carbenicillin indanyl, mezlocillin, piperacillin, ticarcillin, amoxicillin-clavulanic acid, ampicillin-sulbactam, benzylpenicillin, cloxacillin, dicloxacillin, methicillin, oxacillin, penicillin G, penicillin V, piperacillin tazobactam, ticarcillin clavulanic acid, nafcillin, procaine penicillin, cefadroxil, cefazolin, cephalexin, cephalothin, cephapirin, cephradine, cefaclor, cefamandol, cefonicid, cefotetan, cefoxitin, cefprozil, cefmetazole, cefuroxime, loracarbef cefdinir, ceftibuten, cefoperazone, cefixime, cefotaxime, cefpodoxime proxetil, ceftazidime, ceftizoxime, ceftriaxone, cefepime, azithromycin, clarithromycin, clindamycin, dirithromycin, erythromycin, lincomycin, troleandomycin, cinoxacin, ciprofloxacin, enoxacin, gatifloxacin, grepafloxacin, levofloxacin, lomefloxacin, moxifloxacin, nalidixic acid, norfloxacin, ofloxacin, sparfloxacin, trovafloxacin, oxolinic acid, gemifloxacin, pefloxacin, imipenem-cilastatin, meropenem, and aztreonam. In an embodiment, the amount of an antibiotic in a topical composition can be from about 0.01% to about 5% by weight of the total composition.

Antiseptic agents include, but are not limited to, iodine, manuka honey, octenidine dihydrochloride, phenol, polyhexanide, sodium chloride, sodium hypochlorite, calcium hypochlorite, sodium bicarbonate, methyl paraben, benzoyl peroxide, and sodium dehydroacetate. In an embodiment, the amount of an antiseptic agent in the topical formulation can be from about 0.01% to about 5% by weight of the total composition.

Antifungal agents include, but are not limited to, amphotericin B, candicidin, filipin, hamycin, natamycin, nystatin, rimocidin, bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, fluconazole, isavuconazole, itraconazole, posaconazole, ravuconazole, terconazole, voriconazole, abafungin, amorolfin, butenafine, naftifine, terbinafine, anidulafungin, caspofungin, micafungin, benzoic acid, ciclopirox, flucytosine, griseofulvin, haloprogin, tolnaftate, undecylenic acid, crystal violet, and balsam of Peru. In an embodiment, the amount of an antifungal agent in the topical formulation can be from about 0.01% to about 5% by weight of the total composition.

Analgesic agents include, but are not limited to, methyl salicylate, codeine, morphine, methadone, pethidine, buprenorphine, hydromorphine, levorphanol, oxycodone, fentanyl, and a non-steroidal anti-inflammatory drug. In an embodiment, the amount of an analgesic agent in a topical formulation can be from about 0.01% to about 5% by weight of the total composition.

Antiviral agents include, but are not limited to, acyclovir, famciclovir, penciclovir, valacyclovir, trifluridine, docosanol, amantadine, rimantadine, oseltamivir, and zanamivir. In an embodiment, the amount of an anti-viral agent in a topical formulation can be from about 0.01% to about 5% by weight of the total composition.

Stabilizer agents include, but are not limited to, guar gum, xanthan gum cellulose hyaluronic acid, polyvinyl pyrrolidone (PVP), alginate, chondritin sulfate, poly gamma glutamic acid, gelatin, chitisin, corn starch, and flour. In an embodiment, the amount of a stabilizer agent in a topical formulation can from about 0.25% to about 2% (w/v).

Formulations may be prepared by conventional procedures known in the pharmaceutical, cosmetic, skin care, personal care, food, and industrial chemical fields. It can be appreciated that the compositions described herein may be varied according to well-known techniques to accommodate differing amounts and types of ingredients. Additionally, the specific ingredients and proportions described herein are for illustrative purposes. Ingredients may be exchanged for suitable equivalents or substitutes, and proportions may be varied, according to the desired properties of the dosage form of interest.

Many active and inactive ingredients are characterized by low solubility in water and high solubility in oil or lipid mixtures. In topical applications, they do not easily adhere to or remain on skin, and have limited penetration properties. Some ingredients cannot be taken orally (due to ineffectiveness, toxicity issues or other adverse side effects). Given these limitations, it is challenging for a formulator to find the best (active and inactive) ingredients, and the best loading requirements, to enhance the efficacy of the ingredients in the composition when it is taken orally or applied to and comes in contact with the skin of a human or an animal. Similar limitations need to be addressed in other dosage formats (ocular, mucosal, intravenous, intramuscular, sublingual, etc.)

A skilled artisan in the field of topical formulations can utilize the compositions and methods described herein to create a high-quality product. The compositions according to the invention may be applied to any part of the skin, such as the face, scalp, back and other parts of the body. Once in contact with the skin, the inventive compositions can effectively and efficiently deliver an active and/or inactive ingredient, and provide its beneficial attributes to a human or animal in need thereof.

The invention includes a method for treating skin disorders and other types of ailments by applying a therapeutically effective amount of the inventive compositions for a therapeutically effective amount of time to the skin of a human or an animal in need of treatment thereof. When the composition comes in contact with the skin, an active ingredient contained therein can treat or ameliorate a variety of skin disorders and other types of diseases and ailments (e.g., systematic inflammation).

The skin Is the largest organ that covers and protects the body. Skin disorders (i.e., diseases or ailments) are conditions that affect a person's skin. The disorders may produce rashes, inflammation, itchiness, skin damage or other detrimental changes to the skin. The cause for skin conditions may be due to genetic and/or lifestyle factors. Skin disease treatment may include medications, creams, ointments, or lifestyle changes. Skin ailments include conditions that clog, irritate or inflame the skin to cause rashes or other changes in the skin's appearance. Some skin illnesses are minor, while others cause severe symptoms. Some of the more common skin diseases include:
Acne—blocked skin follicles that lead to oil, bacteria and dead skin buildup in pores.
Alopecia areata—loss of hair in small patches.
Atopic dermatitis (eczema)—dry, itchy skin that leads to swelling, cracking or scaliness.
Psoriasis—scaly skin that may swell or feel hot.
Raynaud's phenomenon—periodic reduced blood flow to fingers, toes or other body parts, causing numbness or skin color change.
Rosacea—flushed, thick skin and pimples, usually on the face.
Skin cancer—uncontrolled growth of abnormal skin cells.
Vitiligo—patches of skin or hair that contain less pigment than surrounding skin or hair areas.
Melasma—patches of (brown or grey-brown) skin that contain more pigment than surrounding skin areas.
Varicose or spider veins—enlarged, twisted damaged veins, often found close to the skin surface on the legs.

Common Causes of Skin Diseases Include:
Bacteria trapped in pores or hair follicles.
Conditions that affect your thyroid, kidneys or immune system.
Contact with environmental triggers causing inflammation, such as allergens (e.g., poison ivy and poison oak) or contact with the skin of another person or animal.
Genetic disorders and/or Hormone disorders.
Fungus or parasites living on skin.
Medications, such as the sulfasalazine, a drug used to block inflammation in Crohn's disease, may cause an allergic skin rash, hives, or itching.
Viruses, such as human papilloma virus (HPV) or herpes simplex virus (HSV).
Conditions related to diabetes, such as diabetic dermopathy, necrobiosis lipoidica diabeticorum, diabetic blisters, and eruptive xanthomatosis.

Some of the Examples shown below provide an optimized topical oil-free aqueous emulsion system for the delivery of fisetin, trans-resveratrol and/or milk thistle extract comprising silymarin to promote hair growth. The formulation can be used to treat alopecia or for any other reason to promote hair growth (e.g., growing hair on shaved areas of the body after a medical procedure has been performed).

Other Examples shown below demonstrate the wide range of topical applications of the formulation technology using:
fisetin as a hair growth (and hair loss prevention) treatment.
quercetin and (−)-epigallocatechin-3-gallate (EGCG) for the treatment of wart masses (e.g., common, plantar and flat) and cold sores.
curcumin for the treatment of inflammation.
salicylic acid for acne treatment.
Centipeda minima (an extracting herb) for the treatment of hair loss, eczema, or other skin conditions involving inflammation.
dihydroquercetin for hair growth treatment and hair loss prevention.
Rutin and trans-resveratrol, and optionally, diosmin for the treatment of varicose veins.
Imatinib or imatinib mesylate for the treatment of pigmentary disorders (e.g., vitiligo and melasma).

Hair growth and hair loss prevention with an inventive formulation may occur via different ways. An inventive formulation can slow down the rate of hair loss and/or promote the growth of new hair. It may repair/reactivate inactive hair follicles and/or grow new hair follicles. It may: (i) slow down, halt and/or reverse the miniaturization of hair follicles; (ii) increase the blood flow around hair follicles; (iii) stimulate follicle movement to a growth phase; and/or (iv) extend the growth phase of hair follicles. An inventive formulation can promote hair growth rates of a quarter, half, three-quarter, or one inch per month, or any rate in between. The growth rate can result in an increase of the concentration of hair in the treated area of about 25%, about 50%, about 75%, about 100%, or any amount in between.

Wart resolution with an inventive formulation may occur by shedding layers of the wart mass until it is resolved. An inventive formulation can be applied to a wart mass until the mass is no longer visible and/or the area looks the same as the surrounding skin. Black dots or areas of grainy texture that were present are not visible anymore. The wart resolution may be about 25%, about 50%, about 75%, about 100%, or any amount in between.

A commonly used synthetic emulsifying agent found in a variety of products is a polyethylene glycol (PEG: H(OCH₂CH₂)ₙOH) derivative of castor oil, where n equals 2 to about 1,000. For instance, PEG 40 hydrogenated castor oil (a PEG derivative of castor oil abbreviated as PEG 40 HCO) serves as a multi-functional emulsifier, surfactant, foam booster, emollient, cleaning agent, solubilizer, thickener, and/or co-solvent. It can be found in many goods spanning various industries, such as pharmaceuticals, cosmetics, food products, skin care products, and personal care products. PEG 40 HCO does not possess oil properties. It does, however, possess exceptional emulsifying and solubilizing properties, and is soluble in both water and oil. It is particularly useful in aqueous formulations containing a large water phase, and in combination with an oil or other type of fat or lipid, can prepare oil-in-water nano-emulsions having clear and stable oil droplets.

The Examples below utilized PEG 40 HCO to formulate an aqueous emulsion without the presence of an oil phase. While the Examples employed PEG 40 HCO as an emulsifier, the emulsions of the invention can also be made with various other grades of polyoxyl n hydrogenated castor oil grades, for example, those where n equals about 30 to about 40 in the PEG chain.

Oleic acid is a fatty acid that occurs naturally in various animal and vegetable fats and oils. It is classified as a monounsaturated omega-9 fatty acid. Oleic acid is used as a component in many foods, in the form of its triglycerides. It is a component of the normal human diet, being found in animal fats and vegetable oils. Oleic acid as its sodium salt is a major component of soap as an emulsifying agent. It is also used as an emollient. Small amounts of oleic acid are often used as an excipient in pharmaceuticals, or an emulsifying or solubilizing agent in aerosol products. Oleic acid is widely used in the solution phase synthesis of nano-particles, functioning as a kinetic knob to control the size and morphology of nanoparticles.

A commonly used co-solvent for solubilizing a chemical compound is diethylene glycol monoethyl ether [‘DEGEE’, also known as 3,6-dioxa-1-octanol, 2-(2-ethoxyethoxy)ethanol, ethoxydiglycol, or by the trade name Transcutol®]. It can be manufactured by the condensation of ethylene oxide and ethanol, followed by distillation. The presence of both ether and alcohol functionalities in the molecule provides excellent solubilizing capacity. DEGEE is an odorless liquid that is used chiefly as a solubilizer and permeability enhancer for making a variety of products, including soaps, dyes, cosmetics (e.g., vitamin C, dihydroxyacetone (DHA) and benzoyl peroxide), and other chemicals. It has also been used in hair care products to promote a longer lasting and more uniform coloring. There are limitations for the usage of DEGEE. DEGEE cannot be used as the sole solvent (100%) and generally needs to be formulated to a concentration of less than about 50% (preferably, less than about 20%) in a topical formulation. (For example, in the FDA Inactive Ingredient Database, there is an FDA approved topical product using 49% DEGEE.) While DEGEE does not possess any oil or emulsification properties, it is a useful co-solvent for its miscibility properties with water and some oils, like almond oil or oleic acid.

Oil-in water (o/w) and water-in-oil (w/o) emulsions comprising oil, water, surfactants (or other emulsifying agents), and co-solvents are frequently formulated to improve the look, delivery, and bioavailability of an active compound(s). DEGEE and PEG 40 HCO have been used to make o/w emulsions with some of the compounds described herein. Most emulsions contain particles and droplets having a mean diameter of > about 1 μm. However, mini-, micro-, and nano-emulsions according to the invention can be designed with particle and droplet sizes in the range of about 100-500 nm. With proper formulation, highly stable emulsions can be prepared having particles and droplets as small as a few nanometers.

As described above, the formulations of the invention can be useful to treat a variety of skin disorders, such as alopecia (hair loss). Humans and animals that suffer from hair loss is widespread. There are many types of treatment that may reverse hair loss, or at least slow it down. The treatments include medications and surgery. Two popular medications are minoxidil (Rograine®) and finasteride (Propecia©). Minoxidil is applied topically and finasteride is taken orally. Minoxidil has several side effects (e.g., scalp irritation and unwanted hair growth on the adjacent skin of the face and hands) and must be discontinued if a rash appears. Orally dosed finasteride suffers from rare side effects (e.g., diminished sex drive and sexual function and an increased risk of prostate cancer) and is contraindicated in pregnant women because of the risk of hypospadias in male fetuses. Other oral medications include spironolactone (Carospir®, Aldactone®) and oral dutasteride (Avodart®). Other topical products that have been studied are based on natural products that contain biotin, amino acids, curcumin, and caffeine as growth aids. Various other tinctures, serums and mixtures of natural products have also been studied and sometimes marketed for hair loss.

Notwithstanding the various types of treatment for hair loss, their effectiveness, and their side effects, are less than desirable. There remains a large need for a more effective hair loss product. Some hair growth studies in the literature have sought to find new hair growth compounds that work through biological pathways that are different than the pathways employed by today's commercial products (which are generally based on the classic androgenic hair loss model). Recent hair growth compounds include fisetin, trans-resveratrol, and silymarin (from milk thistle extract). These compounds are natural products that have been demonstrated to promote growth by the JAK/STAT and Wnt/beta-catenin molecular pathways. However, notwithstanding the promise of the growth promotion properties, it has been reported that the compounds when dosed as solutions were found not to be optimal for long term applications (e.g., ethanol and isopentyl diol toxicity and side effects). Fisetin delivered topically with an ethanol-based formulation was reported to be not viable because it damaged hair, while trans-resveratrol topically delivered with an isopentyl diol formulation was reported to block hair follicles. Accordingly, these formulation vehicles are generally not acceptable for a commercial product.

Emulsion technology has been applied to numerous active molecules, including fisetin, trans-resveratrol and silymarin (from milk thistle extract). Fisetin, trans-resveratrol, and silymarin are active constituents of natural products that have been the subject of numerous studies accessing their hepatoprotective, antioxidant, anti-inflammatory, immunomodulatory, anti-cancer, cardioprotective, and hair growth properties. However, the studies have shown that all three compounds possess low oral bioavailability, thus limiting their effectiveness. Consequently, the three active compounds have not been dosed orally and various formulations have been developed to topically deliver them to the body. Several studies have shown that some of the compounds can be topically delivered, predominantly through the use of oil-in-water emulsion technology.

The structures of fisetin, trans-resveratrol, and silymarin are shown in Scheme 1 below.

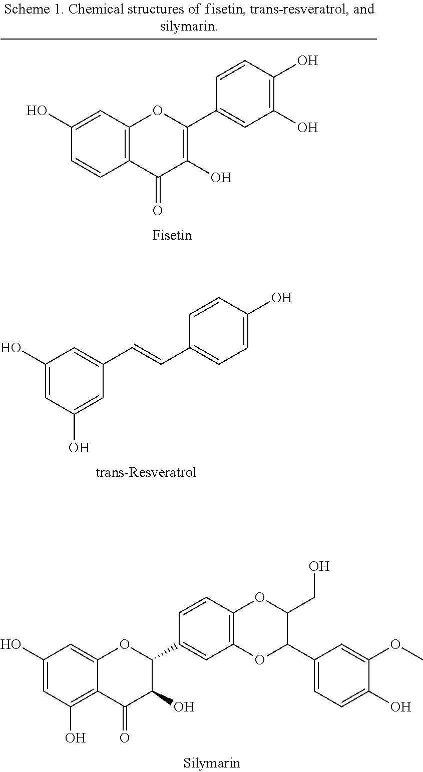 [SMILES: O=c1c(O)c(-c2ccc(O)c(O)c2)oc2cc(O)ccc12 ; Oc1ccc(/C=C/c2cc(O)cc(O)c2)cc1 ; COc1cc(C2Oc3cc([C@H]4Oc5cc(O)cc(O)c5C(=O)[C@@H]4O)ccc3OC2CO)ccc1O]

Fisetin (3,3′,4′,7-tetrahydroxyflavone) (2-(3,4-dihydroxyphenyl)-3,7-dihydroxy-4H-1-benzopyran-4-one) (Scheme 1) is a bioactive flavonol (flavonoid group of polyphenols) molecule found in many plants, fruits and vegetables, such as strawberry, apple, persimmon, grape, onion, and cucumber at concentrations in the range of about 2-160 μg/g. It has been suggested that fisetin possesses antiproliferative, apoptotic, and/or antioxidant activities. Papers have reported that fisetin may be an effective chemopreventive/chemotherapeutic agent in several types of cancer and may have neuroprotective properties. However, fisetin has also been reported to have low oral bioavailability, likely due to its poor aqueous solubility (10.45 mcg/mL) and high lipophilicity (log P 3.2). Additionally, fisetin has been shown to be highly metabolized when taken orally or intravenously.

Resveratrol (5-[(E)-2-(4-hydroxyphenyl)ethen-1-yl]benzene-1,3-diol) (Scheme 1) is a natural polyphenolic stilbenoid found in a variety of plants, including many food items, such as blueberries, mulberries, raspberries, grapes, red wine, and peanuts. It exists as two isomers, namely, cis- and trans-resveratrol. The trans isomer has been reported as the more biologically active compound. Classified as a phytoalexin, trans-resveratrol can be produced in response to a fungal attack or the onset of certain types of stress, including trauma and UV irradiation. Trans-resveratrol has been examined for its potential therapeutic benefit in the treatment or prevention of cancer, heart disease, neurodegeneration, and inflammation. Trans-resveratrol is generally produced by isolating it from extracts of red wine/grape skin or from the plant Polygonum cuspidatum. However, it has low oral bioavailability, and its utility as a therapeutic in humans has been limited. The bioavailability of trans-resveratrol is about 0.5%, likely due to extensive hepatic glucuronidation and sulfation. The viability of an oral delivery method with trans-resveratrol is questionable due to its low aqueous solubility and short half-life (about 15 min).

Silymarin (Scheme 1) is a flavonolignan extracted from the milk thistle Silybum marianum (L.) gaernt plant. It is composed of an isomeric mixture of seven flavonolignans—silybin A, silybin B isosilybin A, isosilybin B, silychristin A, silychristin B, and silydianin—and one flavonoid—taxifolin. Silybin, also known as silibinin, has the IUPAC chemical name of (2R,3R)-3,5,7-trihydroxy-2-[(2R,3R)-3-(4-hydroxy-3-methoxyphenyl)-2-(hydroxymethyl)-2,3-dihydrobenzo[b][1,4]dioxin-6-yl]chroman-4-one. Silymarin has been shown to possess various pharmacological properties, like hepatoprotective, antioxidant, anti-inflammatory, anti-cancer, anti-fibrotic, and cardioprotective activities. Although clinical trials have shown silymarin to be non-toxic in humans at high doses (> about 1500 mg/day), pharmacokinetic studies have revealed poor absorption, rapid metabolism, and ultimately, poor oral bioavailability of the molecule.

There are many cases of oil-in-water mini-, micro-, and nano-emulsions in the literature. See e.g., U.S. Pat. Nos. 10,918,606 and 10,736,842, and US Patent Publication Nos. 2021/0330598, 2021/077396, 2021/0046087, and 2018/0071390, which are hereby incorporated by reference in their entirety. Oil-in-water emulsions have been studied with fisetin, trans-resveratrol and silymarin, as well as other active chemical compounds, such as quercetin or dihydroquercetin (a flavonoid in the same class of compounds as fisetin, silymarin, and silibinin), carvediol, nitrendipine, vinpocetine, curcumin, (−)-epigallocatechin-3-gallate (a polyphenol abbreviated as EGCG), and salicylic acid.

The molecular structures of quercetin, curcumin, (−)-epigallocatechin-3-gallate, salicylic acid, and rutin are shown in Scheme 2 below.

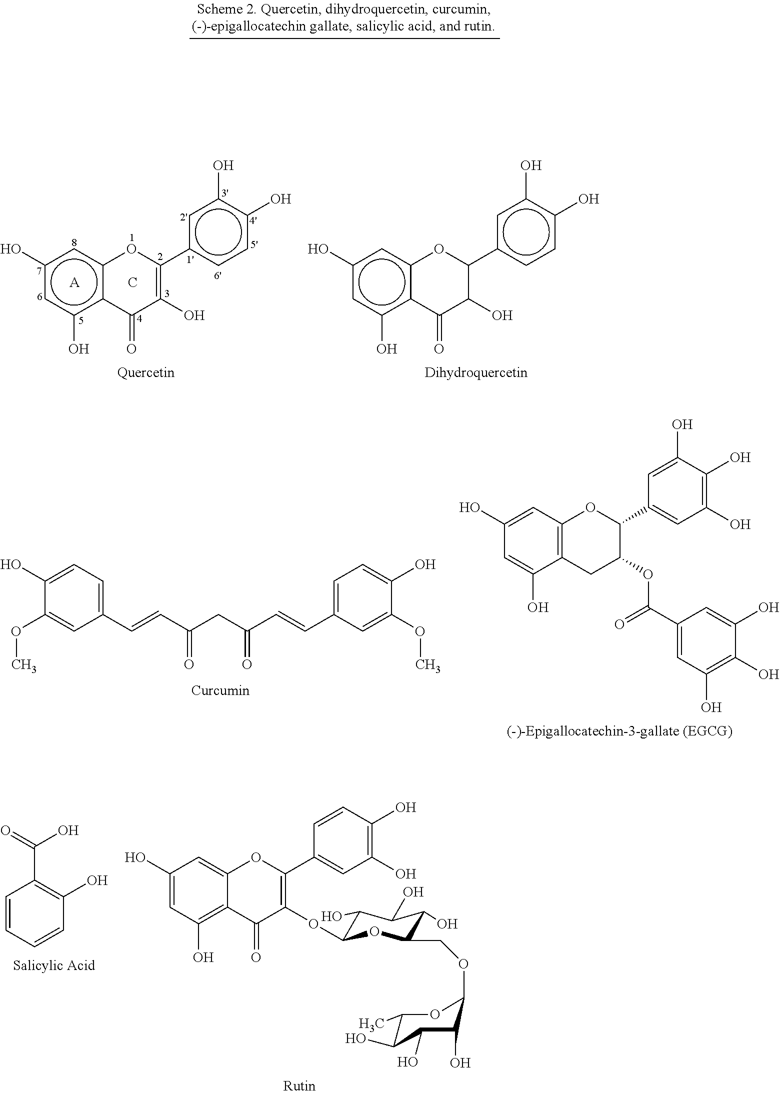

Quercetin (2-(3,4-dihydroxyphenyl)-3,5,7-trihydroxy-4H-1-benzopyran-4-one) and dihydroquercetin are plant pigment flavonoids (flavonols) (Scheme 2). They are potent antioxidants found in many plants and foods, such as red wine, onions, asparagus, broccoli, buckwheat, green tea, grapes, apples, cherries, berries, and citrus fruits. Quercetin has been reported to possess protective abilities against tissue injury induced by various drug toxicities and exhibit antioxidant, anti-inflammatory, anti-bacterial, and anti-viral properties that might help reduce swelling, kill cancer cells, control blood sugar, and help prevent heart disease. It has been used as a prophylactic for oxidative stress diseases (cardiovascular, bronchopulmonary, etc.). Dihydroquercetin has been reported to be more potent and bioavailable than quercetin.

Curcumin (1,7-bis(4-hydroxy-3-methoxyphenyl)-1,6-heptadiene-3,5-dione) is a plant-derived polyphenolic carotenoid compound (Scheme 2). It is naturally present in the rhizome of the yellow root plant Curcuma longa (turmeric) and other Curcuma spp. It has been the subject of intensive investigations on account of its various activities (e.g., anti-inflammatory, anti-cancer, etc.) and can be found in numerous oral herbal supplements. Curcumin may help in the management of exercise-induced inflammation and muscle soreness, thus enhancing recovery and performance in active people. Dosing curcumin orally generally does not lead to the associated health benefits due to its poor bioavailability, which appears to be primarily due to poor absorption, rapid metabolism, and rapid elimination. Essential Turmeric Oil (ETO) coated curcumin has been shown to have about 7 to 10-fold higher bioavailability and be retained longer in systemic circulation, compared with standard curcumin.

(−)-Epigallocatechin-3-gallate ([(2R,3R)-5,7-dihydroxy-2-(3,4,5-trihydroxyphenyl)chroman-3-yl]3,4,5-trihydroxybenzoate) (EGCG) is an ester of epigallocatechin and gallic acid (Scheme 2). It is a type of catechin. EGCG is a polyphenol (flavanol). It is the most abundant catechin in green tea. It has been studied for its potential to affect human health and diseases due to its antibacterial, antiviral, and anti-cancer properties. However, when taken orally, EGCG has poor absorption even at daily intakes equivalent to about 8-16 cups of green tea. After consumption, EGCG blood levels usually peak within 1.7 hours. The absorbed plasma half-life is about 5 hours, but with a majority of unchanged EGCG being excreted into the urine from almost immediately to about 8 hours.

Salicylic acid (2-hydroxybenzoic acid) belongs to a class of drugs known as salicylates (Scheme 2). When applied to the skin, salicylic acid may work by helping the skin to shed dead cells from the top layer and by decreasing redness and swelling (inflammation). This application can decrease skin disorders, such as acne and warts, and speed the healing of the skin by ramping up the body's immune system.

Rutin (2-(3,4-dihydroxyphenyl)-5,7-dihydroxy-3-[(2S,3R,4S,5S,6R)-3,4,5-trihydroxy-6-[[(2R,3R,4R,5R,6S)-3,4,5-trihydroxy-6-methyloxan-2-yl]oxymethyl]oxan-2-yl]oxychromen-4-one) (Scheme 2), also known as rutoside, quercetin-3-rutinoside, and sophorin, is the glycoside combining the flavonol quercetin and the disaccharide rutinose. It is a flavonoid found in a wide variety of plants, fruits, and vegetables (e.g., the citrus flavonoid glycoside found in buckwheat). Rutin may have antioxidant and anti-inflammatory effects. It has been studied as a potential prophylactic against cancer and other diseases. Rutin is commonly used for autism, aging skin, airway infections caused by exercise, and other purposes, but there is little scientific evidence to support its effectiveness.

Centipeda minima (Kshavak/Sneezewort) is a plant leaf herb that has been used in Chinese medicine to treat respiratory diseases for centuries. Twelve common components, including flavones and their glycosides, phenolic and polyphenolic acids, and sesquiterpene lactone have been identified in an ethanol extract of Centipeda minima. Brevilin A is a sesquiterpene lactone isolated from Centipeda minima that has been shown to inhibit Janus kinase 3 (JAK3), which is a cellular pathway implicated in hair loss and cancer treatment.

Other active ingredients, such as diosmin, imatinib, and imatinib mesylate can be utilized in the aqueous emulsions described herein. Their molecular structures are shown in Scheme 3 below.

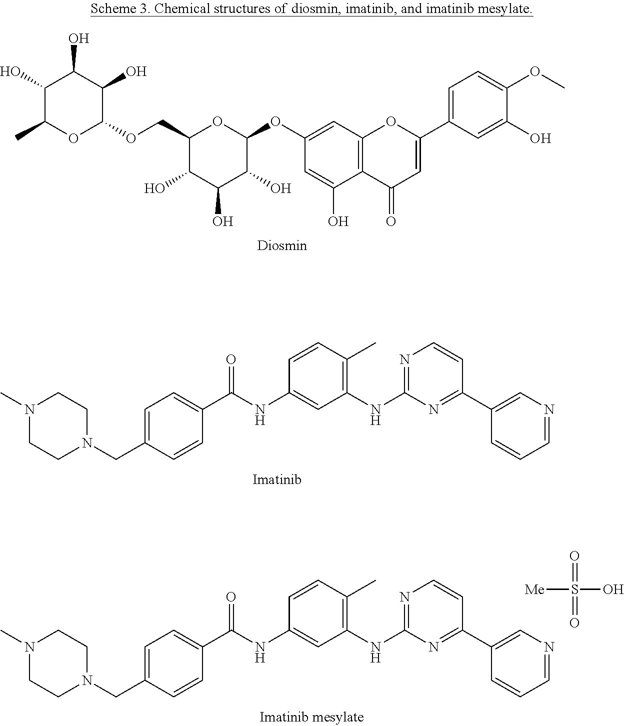

Diosmin(5-hydroxy-2-(3-hydroxy-4-methoxyphenyl)-7-[(2S,3R,4S,5S,6R)-3,4,5-trihydroxy-6-[[(2R,3R,4R,5R,6S)-3,4,5-trihydroxy-6-methyloxan-2-yl]oxymethyl]oxan-2-yl]oxychromen-4-one) (Scheme 3) is a disaccharide derivative that consists of diosmetin substituted by a 6-O-(alpha-L-rhamnopyranosyl)-beta-D-glucopyranosyl moiety at position 7 via a glycosidic linkage. It has a role as an antioxidant and an anti-inflammatory agent. It is a glycosyloxyflavone, a rutinoside, a disaccharide derivative, a monomethoxyflavone and a dihydroxyflavanone. It is functionally related to diosmetin.

Imatinib (4-[(4-methylpiperazin-1-yl)methyl]-N-(4-methyl-3-{[4-(pyridin-3-yl)pyrimidin-2-yl]amino}phenyl)benzamide) and its mesylate salt (Scheme 3) are 2-phenyl amino pyrimidine derivatives that function as specific inhibitors of a number of tyrosine kinase enzymes. They are small molecule inhibitors targeting multiple receptor tyrosine kinases such as CSF1R, ABL, c-KIT, FLT3, and PDGFR-β. Imatinib mesylate is commercially available under the brand names Gleevec and Glivec, among others, and is used as an oral targeted therapy medication to treat cancer.

The above compounds were formulated using the inventive emulsion system and topical compositions were applied to the skin of a human subject to test a variety of disorders.

The new compositions described herein comprise a poorly water-soluble chemical compound in an aqueous based emulsion that substantially or completely does not possess an oil phase. Exemplary compounds include, but are not limited to, one or more of the compounds shown in Schemes 1 and 2 above. The compositions are useful for topical and other modes of application to treat various skin disorders (e.g., hair loss, plantar warts, cold sores, etc.) and other types of diseases or ailments.

The following Examples are intended to illustrate the invention and should not be construed as to narrow its scope. One skilled in the art will readily recognize that the Examples suggest other ways in which the invention could be practiced. It should be understood that numerous variations and modifications may be made while remaining within the scope of the invention.

### EXAMPLES

### Example 1. Formulations of a Topical Composition Comprising Fisetin, Trans-Resveratrol, and Milk Thistle (Comprising Silymarin)

This study sought to formulate active ingredients fisetin, trans-resveratrol, and milk thistle extract (comprising silymarin) together in a combination product, which could be useful as a topical composition to prevent hair loss and/or promote hair growth. The study delivered a stable and transparent oil-free aqueous nano-emulsion comprising the active ingredients, polyethylene glycol (PEG) 40 hydrogenated castor oil (PEG 40 HCO), diethylene glycol monoethyl ether (DEGEE), and water, where the active ingredients were dispersed in an oil-free topical emulsion.

DEGEE is an odorless liquid co-solvent that is miscible in water and can enhance the solubility and permeability of chemical compounds when they are applied to the skin of a human or animal.

PEG 40 HCO is a waxy, viscous liquid emulsifier that is miscible in water. PEG 40 HCO by itself did not effectively dissolve fisetin, trans-resveratrol or milk thistle extract (comprising silymarin) powders. It did not sufficiently wet the compound powders and was too viscous to act as a solvent by itself. However, when PEG 40 HCO was added to a DEGEE mixture containing fisetin, trans-resveratrol, and milk thistle extract (comprising silymarin), dissolution of all three compounds was successfully accomplished.

Fisetin, trans-resveratrol, and milk thistle extract (comprising silymarin) were all found to be soluble in DEGEE in a solubility screening panel evaluating surfactants and other emulsifiers for use in topical delivery. An initial objective was to find a suitable miscible oil for use in hair applications, such as argan, sesame, sunflower, fractionated coconut, and grape seed oils. None of the evaluated oils were miscible with DEGEE and the resulting mixtures exhibited visible separation of DEGEE and an oil phase. The next step was to evaluate oil soluble emulsifiers, such as glyceryl oleate, sorbitan oleate, lauryl oleate, and lauryl sulfate. Glyceryl oleate was found to increase the miscibility of DEGEE with argan oil. However, when the mixture was diluted with water, a suspension formed, and the active ingredients precipitated out of solution. Oleic acid (a fatty acid) is known to be miscible with DEGEE, but it is a non-comedogenic fatty acid, which makes it an unfavorable choice for use in a long-term topical dosing formulation. Oleic acid was evaluated with a mixture of sorbitan laurate, polyglyceryl-4 laurate, and dilauryl citrate. This formulation made a miscible mixture with fisetin, trans-resveratrol, and milk thistle dissolved in DEGEE. However, the dilution of the mixture with water created a suspension, and once again, the solids precipitated out after approximately one hour.

At this point, the objective of the study changed. Instead of further evaluating mixtures for an oil phase, it was decided to directly dilute the mixture with water to see what would happen. It was expected that further water dilution would cause the active ingredients that were tested to precipitate out. However, instead of precipitation of the active ingredients, the diluted solution held a clear yellow appearance and remained stable. Without being bound by any particular theory, it is posited that this result indicates that either a nano-emulsion was formed, or PEG 40 HCO somehow created a supersaturated solution. The outcome was surprising and unexpected because the previous formulations included an oil (or fatty acid) ingredient to create an oil-in-water emulsion, but this formulation lacked an oil phase as a component of the mixture, yet produced a stable and clear aqueous emulsion.

Given the surprising result of generating an oil-free aqueous emulsion, the following formulations (A-H) were prepared:

A—Fisetin dissolved in DEGEE at about 0.15%, then diluted 1:1 with water.

B—trans-Resveratrol dissolved in DEGEE at about 0.15%, then diluted 1:1 with water.

C—Milk thistle extract (comprising silymarin) dissolved in DEGEE at about 0.25%, then diluted 1:1 with water.

D—about 0.15% fisetin, about 0.15% trans-resveratrol, about 0.25% milk thistle extract (as about 0.2% silymarin), about 10% PEG 40 hydrogenated castor oil (PEG 40 HCO), about 20% diethylene glycol monoethyl ether (DEGEE), and about 69.45% purified water.

E—about 0.075% fisetin, about 0.075% trans-resveratrol, about 0.125% milk thistle extract (as about 0.1% silymarin), about 5% PEG 40 hydrogenated castor oil (PEG 40 HCO), about 10% diethylene glycol monoethyl ether (DEGEE), and about 84.725% purified water. (further dilution of formulation D)

F—about 0.15% fisetin, about 0.15% trans-resveratrol, about 0.25% milk thistle extract (as about 0.2% silymarin), about 5% PEG 40 hydrogenated castor oil (PEG 40 HCO), about 10% diethylene glycol monoethyl ether (DEGEE), and about 84.45% purified water.

All mixtures (formulations A-F) were stable and transparent.

G—about 0.15% fisetin, about 0.15% trans-resveratrol, about 0.25% milk thistle extract (as about 0.2% silymarin), about 10% PEG 40 hydrogenated castor oil (PEG 40 HCO), about 10% diethylene glycol monoethyl ether (DEGEE), and about 79.45% purified water.

Formulation G remained stable and transparent and was analyzed for nano-particles (see Table I below). The nano-particle tracking technique observed nano-particles in the mixture with a d50 particle size of about 119.5 nm and a number weighted mean particle size of about 125.4 nm. This result was not expected for the mixture. A person skilled in the art of formulation development would have expected the active ingredients in the PEG 40 HCO and DEGEE mixture without the presence of an oil phase to precipitate out upon dilution with water.

TABLE I
NANOPARTICLE TRACKING ANALYSIS DATA SUMMARY

| | | | | NUMBER | NUMBER | |
|---|---|---|---|---|---|---|
| | CUMULATIVE NUMBER % | | | WEIGHTED | WEIGHTED | STANDARD |
| | LESS THAN INDICATED SIZE (nm) | | | MEAN | MODE | DEVIATION |
| SAMPLE ID | D10 | D50 | D90 | (nm) | (nm) | (nm) |

Ethoxydiglycol + Water

| | | | | | | |
|---|---|---|---|---|---|---|
| 13701-25B | 38.7 | 119.5 | 195.8 | 125.4 | 84.6 | 73.1 |

H—about 0.15% fisetin, about 0.15% trans-resveratrol, about 0.25% milk thistle extract (as about 0.2% silymarin), about 5% PEG 40 hydrogenated castor oil (PEG 40 HCO), about 5% diethylene glycol monoethyl ether (DEGEE), and about 89.45% purified water. Formulation H remained transparent and stable and the nano-particle tracking technique observed nano-particles in the mixture with a d50 particle size of about 37.8 nm and a number weighted mean particle size of about 36.4 nm (see Table II below). These results demonstrate that particle size (and droplet size) can be engineered by the formulation composition. Since a smaller particle size (and droplet size) should theoretically enhance the appearance and permeability of the formulation, it is postulated that formulation H is likely a better candidate than formulation G for dosing as a combination product.

TABLE II
NANOPARTICLE TRACKING ANALYSIS DATA SUMMARY

| | | | | NUMBER | NUMBER | |
|---|---|---|---|---|---|---|
| | CUMULATIVE NUMBER % | | | WEIGHTED | WEIGHTED | STANDARD |
| | LESS THAN INDICATED SIZE (nm) | | | MEAN | MODE | DEVIATION |
| SAMPLE ID | D10 | D50 | D90 | (nm) | (nm) | (nm) |

Ethoxydiglycol + Water

| | | | | | | |
|---|---|---|---|---|---|---|
| 13701-25D | 17.2 | 37.8 | 49.5 | 36.4 | 25.9 | 15.5 |

Nano-sized particles were measured with a Malvern NanoSight NS300. The NanoSight NS300 uses the technology of Nanoparticle Tracking Analysis (NTA). This technology utilizes the properties of both light scattering and Brownian motion in order to obtain the size distribution and concentration measurement of particles in liquid suspension. A laser beam is passed through the sample chamber, and the particles in suspension in the path of this beam scatter light in such a manner that they can easily be visualized via 20× magnification microscope onto which is mounted a camera. The camera operates at 30 frames per second (fps), capturing a video file of the particles moving under Brownian motion. The software tracks many particles individually and using the Stokes-Einstein equation calculates their hydrodynamic diameters. The measurement is controlled via Standard Operating Procedures that are set with a user-friendly software interface.

The following formulations I-M were next prepared.

A base composition comprising about 0.15% fisetin, about 0.15% trans-resveratrol, about 0.25% milk thistle extract (as about 0.2% silymarin), and about 10% diethylene glycol monoethyl ether (DEGEE), together with the following amounts of PEG 40 hydrogenated castor oil (PEG 40 HCO):
I—about 1% PEG 40 HCO.
J—about 2.5% PEG 40 HCO.
K—about 5% PEG 40 HCO.
L—about 7.5% PEG 40 HCO.
M—about 10% PEG 40 HCO.

Each of formulations I-M was diluted to 100% with purified water. The experiments showed that formulations I and J (containing about 1% and about 2.5% PEG 40 HCO, respectively) did not form nano-emulsions, and solid precipitation of the active ingredients were observed in both of the formulations. In contrast, formulations K-M (containing about 5%, about 7.5%, and about 10% PEG 40 HCO) remained transparent and stable.

### Example 2. Formulations of a Composition Comprising Fisetin

Formulations were made using fisetin as the sole active component. The concentration of fisetin was increased to about 0.25% w/w fisetin with about 2.6% DEGEE, about 8.0% PEG 40 HCO, and about 89.15% purified water in the formulation.

Formulations were prepared by dispensing the required quantity of fisetin and adding it to a suitable sized container. The required quantity of DEGEE was added along with a magnetic stir bar and the mixture was stirred until the solids dissolved. PEG 40 HCO is a semisolid at room temperature and has a melting point of approximately 50° C. It was heated to > about 50° C. and added to the fisetin/DEGEE mixture, and the three ingredients were mixed until uniform. The required quantity of purified water was added to the mixture while stirring to create a transparent nano-emulsion.

A formulation containing about 0.25% w/w fisetin (2.5 mg/mL) represents approximately a 250-fold increase in the stable nano-emulsion concentration over the reported aqueous solubility of about 0.01 mg/mL fisetin.

Another formulation was made using a conditioner base as the diluent instead of water. The breakdown of the formulation (with approximate % w/w) is summarized in Table III below.

TABLE III

| Ingredient | % w/w |
|---|---|
| Fisetin | 0.25 |
| DEGEE | 2.6 |
| PEG 40 HCO | 7.5 |
| Water | 84.2 |
| Hydrolyzed lupine protein | 3.0 |
| Tripeptide-29 | 0.3 |
| Cetrimonium Chloride | 0.5 |
| Panthenol | 0.4 |
| Taurine | 0.2 |
| Proline | 0.1 |
| Preservative | 1.0 |
| | 100 |

The formulations created stable and transparent nano-emulsions. They may be employed via different modes of application for a variety of ailments, such as a topical composition for the promotion of hair growth.

### Example 3. Topical Dosing of a Formulation Comprising Fisetin to Promote Hair Growth

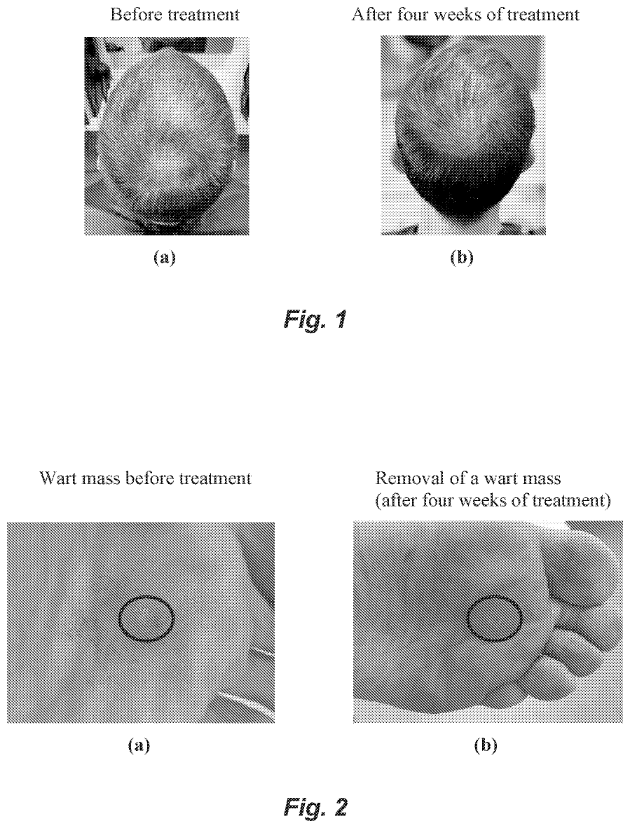

A formulation of Example 2 was evaluated on the scalp of a human male exhibiting hair thinning (alopecia). New hair growth was observed after four weeks of use of a product containing about 0.25% fisetin, about 2.6% DEGEE, about 7.5% PEG 40 HCO, and about 89.65% water. The formulation was applied once daily after a shower and towel drying of hair. The formulation was applied by a dropper and gently massaged into the scalp and allowed to air dry. Approximately 1 mL of product was applied daily. After four weeks of treatment, the male subject was given a haircut and hair growth was evaluated via photographs. Before and after treatment photos are shown in FIGS. 1a and 1b, respectively. Hair growth was observed in the crown region of the scalp after four weeks of treatment. An increase in the concentration of hair present in the treated area looked to be greater than about 25%.

### Example 4. Formulations of a Composition Comprising Trans-Resveratrol

Formulations were made using trans-resveratrol as the sole active component. The concentrations of the ingredients in the formulations were about 0.15% w/w trans-resveratrol, about 2.6% DEGEE, about 7.5% PEG 40 HCO, and about 89.75% purified water.

The formulations were prepared by dispensing the required quantity of trans-resveratrol and adding it to a suitable sized container. The required quantity of DEGEE was added along with a magnetic stir bar and stirred until the solids dissolved. PEG 40 HCO is a semisolid at room temperature and has a melting point of approximately 50° C. It was heated to > about 50° C. and added to the trans-resveratrol and DEGEE mixture, and the three ingredients were mixed until uniform. The required quantity of purified water was added to the mixture while stirring to create a transparent nano-emulsion.

A formulation containing about 0.15% w/w trans-resveratrol (about 1.5 mg/mL) represents approximately about a 50-fold increase in a stable nano-emulsion concentration over the reported aqueous solubility of about 0.03 mg/mL trans-resveratrol.

The formulation can be employed via different modes of application for a variety of ailments, such as a topical composition for the promotion of hair growth.

### Example 5. Formulations of a Composition Comprising Quercetin

Formulations were made using quercetin as the sole active component. Formulations were prepared by dispensing the required quantity of quercetin and adding it to a suitable sized container. The required quantity of DEGEE was added along with a magnetic stir bar and the mixture was stirred until the solids dissolved. PEG 40 HCO is a semisolid at room temperature and has a melting point of approximately 50° C. It was heated to > about 50° C. and added to the quercetin and DEGEE mixture, and the three ingredients were mixed until uniform. The required quantity of purified water was added to the mixture while stirring to create a transparent nano-emulsion.

The following observations were made for each composition:
about 0.5% quercetin, about 2.6% DEGEE, about 8.0% PEG 40 HCO, and about 88.9% water.
   solids precipitated out after 6 hours.
about 0.5% quercetin, about 5.0% DEGEE, about 8.0% PEG 40 HCO, and about 86.5% water.
   precipitated out after 11 hours.
about 0.5% quercetin, about 0.15% fisetin, about 5.0% DEGEE, about 8.0% PEG 40 HCO, and about 86.35% water.
   precipitated out after 11 hours, but 80% less solids than quercetin alone.
about 0.05% quercetin, about 5.0% DEGEE, about 10.0% PEG 40 HCO, and about 84.95% water.
   remained a stable and transparent nano-emulsion.

A formulation containing about 0.5% w/w quercetin (5 mg/mL) represents approximately a 2,500-fold increase in a stable nano-emulsion concentration over the reported aqueous solubility of about 0.002 mg/mL quercetin, albeit for only a few hours. The stable and transparent nano-emulsion formulation containing about 0.05% w/w quercetin (0.5 mg/mL) represents approximately a 250-fold increase over the reported aqueous solubility of quercetin.

The formulation can be employed via different modes of application for a variety of ailments, such as a topical composition for the promotion of hair growth.

### Example 6. Formulations of a Composition Comprising Curcumin

Formulations were made using curcumin as the sole active component. The concentrations of the components in one formulation were about 0.5% w/w curcumin, about 2.6% DEGEE, about 8.0% PEG 40 HCO, and about 88.9% purified water. Another formulation was prepared with about 0.03% w/w curcumin, about 2.6% DEGEE, about 7.5% PEG 40 HCO, and about 89.87% purified water.

The formulations were prepared by dispensing the required quantity of curcumin and adding it to a suitable sized container. The required quantity of DEGEE was added along with a magnetic stir bar and the mixture was stirred until the solids dissolved. PEG 40 HCO is a semisolid at room temperature and has a melting point of approximately 50° C. It was heated to > about 50° C. and added to the curcumin and DEGEE mixture, and the three ingredients were mixed until uniform. The required quantity of purified water was added to the mixture while stirring to create a transparent orange colored nano-emulsion. After approximately 6 hours, solids precipitated out of the solution containing about 0.5% w/w curcumin, while the formulation containing about 0.03% w/w curcumin was stable and transparent and showed no signs of precipitation.

The formulation containing about 0.5% w/w curcumin (about 5 mg/mL) represents approximately an 83,000-fold increase in a stable nano-emulsion concentration over the reported aqueous solubility of 0.0006 mg/mL curcumin, albeit for only a few hours. The formulation containing about 0.03% w/w curcumin (about 0.3 mg/mL) represents approximately a 500-fold increase in a stable nano-emulsion concentration over the reported aqueous solubility of curcumin.

Similar formulations can be crafted along the same lines with turmeric or turmeric/curcumin combinations. The formulation can be employed via different modes of application for a variety of ailments, such as a topical composition to treat inflammation.

### Example 7. Formulations of a Composition Comprising (−)-Epigallocatechin-3-Gallate (EGCG)

Formulations were made using EGCG as the sole active component. The concentrations of the components in the formulations were about 1.0% w/w EGCG, about 5.0% DEGEE, about 10.0% PEG 40 HCO, and about 84.0% purified water.

The formulations were prepared by dispensing the required quantity of EGCG and adding it to a suitable sized container. The required quantity of DEGEE was added along with a magnetic stir bar and stirred until the solids dissolved. PEG 40 HCO is a semisolid at room temperature and has a melting point of approximately 50° C. It was heated to > about 50° C. and added to the EGCG/DEGEE mixture, and the three ingredients were mixed until uniform. The required quantity of purified water was added to the mixture while stirring to create a transparent nano-emulsion.

A formulation containing about 1.0% w/w EGCG (about 10 mg/mL) represents approximately a 2-fold increase in a stable nano-emulsion concentration over the reported aqueous solubility of about 5 mg/mL EGCG.

The formulation can be employed via different modes of application for a variety of ailments, such as a topical composition for the treatment of skin disorders.

### Example 8. Formulations of a Composition Comprising Quercetin and EGCG

Formulations were made using EGCG and quercetin as the active components. The concentrations of the ingredients in one formulation were about 2.0% w/w EGCG, about 0.1% quercetin, about 5.0% DEGEE, about 10.0% PEG 40 HCO, and about 82.9% purified water. The concentrations of the ingredients in another formulation were about 1.0% w/w EGCG, about 0.05% quercetin, about 2.5% DEGEE, about 5.0% PEG 40 HCO, and about 91.45% purified water.

The formulations were prepared by dispensing the required quantity of EGCG and quercetin and adding them to a suitable sized container. The required quantity of DEGEE was added to the EGCG/quercetin mixture along with a magnetic stir bar and the mixture was stirred until the solids dissolved. PEG 40 HCO is a semisolid at room temperature and has a melting point of approximately 50° C. It was heated to > about 50° C. and added to the EGCG/quercetin/DEGEE mixture, and the four ingredients were mixed until uniform. The required quantity of purified water was added to the mixture while stirring to create a transparent nano-emulsion.

The formulation can be employed via different modes of application for a variety of ailments, such as a topical composition for the treatment of skin disorders.

### Example 9. Topical Dosing of a Formulation Containing Quercetin and EGCG

Warts are a type of skin infection caused by a virus (the human papillomavirus (HPV). They can appear anywhere on the body, usually as raised, rough, skin-colored bumps on your skin. There are five major types of warts. Each type appears on a different part of the body and has a distinct appearance. A lesion on the bottom of the foot may be referred to as a plantar wart. This is defined as a wart that grows inward, and can happen as a result of the pressure the feet endure on a daily basis. It can cause severe pain and discomfort.

Salicylic acid, up to about 40% concentration, is a commercially available treatment for warts. Salicylic acid peels the skin away in layers, and removes the wart over time. The acid also irritates (burns) the wart area, which encourages the immune system to respond to the virus. However, it is slow to work and requires frequent application for up to about 12 weeks to effectively treat the skin containing the HPV virus. Freezing off the wart via cryotherapy with liquid nitrogen is another favorable option for many patients, with cure rates of about 50 percent to about 70 percent after three or four treatments.

A formulation comprising EGCG and quercetin was evaluated as an antiviral treatment for the presence of several plantar warts located on the bottoms of the right and left feet of a human male. The warts were treated with a product containing about 0.1% quercetin, about 2.0% EGCG, about 5% DEGEE, and about 10% PEG 40 HCO, in an aqueous system. The formulation was applied twice daily to the affected areas (where the warts were located) once in the morning after a shower and again just before bedtime. The formulation was applied by a cotton swab to the affected area and allowed to air dry.

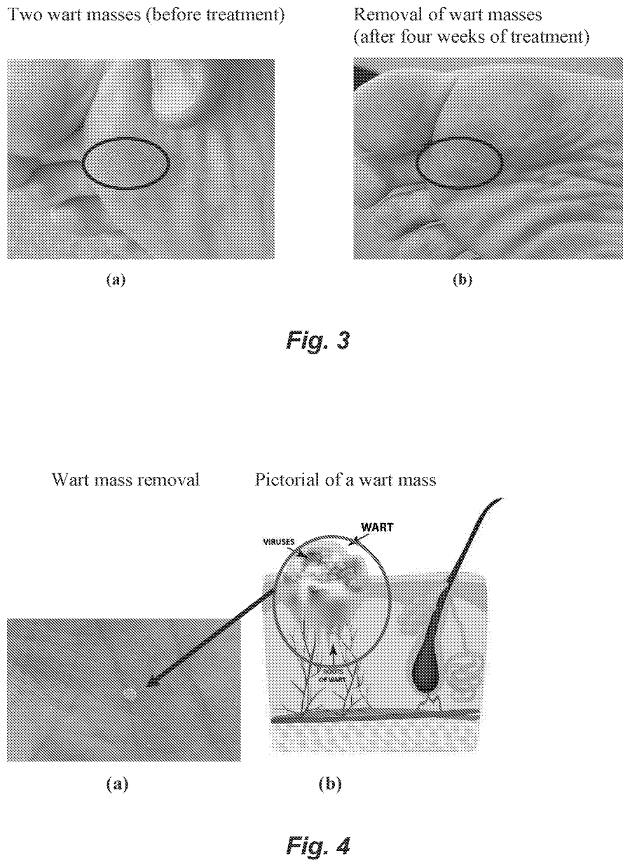

Wart resolution was evaluated via photographs taken after approximately four weeks of treatment. The warts on the feet of the subject male were painful while standing or walking. Within two days of application of the product, the pain had substantially subsided. After about two weeks of application of the product, the viral root of a wart located on the left foot fell off. After about four weeks of application of the product, the viral roots of two warts located on the right foot fell off. Before and after treatments photos at about four weeks showing the absence of viral wart masses after treatment of both feet can be seen in FIGS. 2a and 2b (left foot), respectively, and in FIGS. 3a and 3b (right foot), respectively. The removal of a wart mass from a treated wart mass on a human male foot, and a pictorial showing viruses and roots of a wart are shown in FIGS. 4a and 4b, respectively. The results of the EGCG/quercetin formulation were surprising and suggest that the formulation is acting as an antiviral treatment for warts. It also suggests that the formulation would be similarly effective to treat cold sores.

### Example 10. Formulation of a Composition Comprising Salicylic Acid

A formulation was made using salicylic acid as the sole active component. The concentrations of the components in the formulation were about 0.5% w/w salicylic acid, about 5.0% DEGEE, about 10.0% PEG 40 HCO, and about 84.5% purified water.

The formulation was prepared by dispensing the required quantity of salicylic acid and adding it to a suitable sized container. The required quantity of DEGEE was added along with a magnetic stir bar and the mixture was stirred until the solids dissolved. PEG 40 HCO is a semisolid at room temperature and has a melting point of approximately 50° C. It was heated to > about 50° C. and added to the salicylic acid/DEGEE mixture, and the three ingredients were mixed until uniform. The required quantity of purified water was added to the mixture while stirring to create a transparent nano-emulsion.

A formulation containing about 0.5% w/w salicylic acid (about 5 mg/mL) represents approximately a 2-fold increase in the stable nano-emulsion concentration over the reported aqueous solubility of about 2.48 mg/mL salicylic acid.

The formulation can be employed via different modes of application for a variety of ailments, such as a topical composition for the treatment of skin disorders.

### Example 11. Formulations of Compositions Comprising Milk Thistle (Comprising Silymarin)

A formulation was made using milk thistle (comprising silymarin) as the sole active component. The concentrations of the ingredients in the formulation were about 0.25% w/w milk thistle (comprising about 0.20% silymarin), about 2.6% DEGEE, about 7.5% PEG 40 HCO, and about 89.65% purified water.

The formulation was prepared by dispensing the required quantity of milk thistle and adding it to a suitable sized container. The required quantity of DEGEE was added along with a magnetic stir bar and the mixture was stirred until the solids dissolved. PEG 40 HCO is a semisolid at room temperature and has a melting point of approximately 50° C. It was heated to > about 50° C. and added to the milk thistle/DEGEE mixture, and the three ingredients were mixed until uniform. The required quantity of purified water was added to the mixture while stirring to create a stable and transparent nano-emulsion.

A formulation containing about 0.2% w/w silymarin (2 mg/mL) represents approximately a 5-fold increase in the stable nano-emulsion concentration over the reported aqueous solubility of about 0.4 mg/mL silymarin.

The formulation can be employed via different modes of application for a variety of ailments, such as a topical composition for the promotion of hair growth, vitiligo, and melasma.

### Example 12. Formulations of Compositions Comprising an Extract of Centipeda Minima

A formulation was made using an extract of Centipeda minima as the sole active component, which demonstrated an extraction of an herbal active ingredient with DEGEE. Dried Centipeda minima was milled in a bladed coffee grinder to create a fine powder and added to a suitable sized container. The required quantity of DEGEE along with a magnetic stir bar was added to the milled powder at about a 4:1 extraction ratio and the mixture was stirred until the solids dissolved. The resulting liquid mixture was allowed to stir on a magnetic stir plate for approximately 6 hours. The liquid was collected in a syringe and then filtered through a 1.4 μm glass syringe filter. A dark green, stable and transparent, Centipeda minima extract/DEGEE mixture was collected.

PEG 40 HCO is a semisolid at room temperature and has a melting point of approximately 50° C. It was heated to > about 50° C. and added to the Centipeda minima extract/DEGEE mixture, and the three components were mixed on a magnetic stir plate until uniform. The required quantity of purified water was added to the mixture while stirring to create a transparent nano-emulsion. The concentrations of the components in the nano-emulsion formulation were about 3.0% DEGEE extract mixture, about 7.5% PEG 40 HCO, and about 89.5% purified water. The Centipeda minima extract/DEGEE/PEG 40 HCO nano-emulsion was stable and transparent.

The Centipeda minima extract/DEGEE mixture and the Centipeda minima extract/DEGEE/PEG 40 HCO nano-emulsion were each diluted with water at about a 1:1 ratio. But, the components that were soluble in DEGEE precipitated out of solution when water was added to the Centipeda minima extract/DEGEE mixture forming a cloudy suspension. However, the components in the Centipeda minima extract/DEGEE/PEG 40 HCO nano-emulsion remained stable and transparent.

The nano-emulsion formulation can be employed via different modes of application for a variety of ailments, such as a topical composition for the promotion of hair growth.

### Example 13. Formulation of a Composition Comprising Dihydroquercetin

A formulation was made using dihydroquercetin as the active component. The concentrations of the components in the formulation were about 0.1% w/w dihydroquercetin, about 2.6% DEGEE, about 7.5% PEG 40 HCO, about 88.8% purified water, and about 1.0% preservative.

The formulation was prepared by dispensing the required quantity of dihydroquercetin and adding it to a suitable sized container. The required quantity of DEGEE was added along with a magnetic stir bar and the mixture was stirred until the solids dissolved. PEG 40 HCO is a semisolid at room temperature and has a melting point of approximately 50° C. It was heated to > about 50° C. and added to the dihydroquercetin/DEGEE mixture, and the three ingredients were mixed until uniform. The required quantity of purified water was added to the mixture while stirring to create a transparent nano-emulsion, which was followed by the addition of the preservative.

The formulation can be employed via different modes of application for a variety of ailments, such as a topical composition for the promotion of hair growth or to treat skin disorders.

### Example 14. Formulation of a Composition Comprising Rutin and Trans-Resveratrol

A formulation was made using rutin and trans-resveratrol as the active components. The concentrations of the components in the formulation were about 0.05% w/w rutin, about 0.15% trans-resveratrol, about 2.6% DEGEE, about 5.0% PEG 40 HCO, about 1.0% preservative, about 1.0% xanthan gum, and about 90.2% purified water.

The formulation was prepared by dispensing the required quantity of rutin and resveratrol to a suitable sized container. The required quantity of DEGEE was added to the container along with a magnetic stir bar and the mixture was stirred until the solids dissolved. PEG 40 HCO is a semisolid at room temperature and has a melting point of approximately 50° C. It was heated to > about 50° C. and added to the rutin/trans-resveratrol/DEGEE mixture, and the ingredients were mixed until uniform. The required quantity of purified water was added to the mixture while stirring to create a transparent nano-emulsion followed by the addition of the preservative and xanthan gum.

The formulation can be employed via different modes of application for a variety of ailments, such as a topical composition for the treatment of varicose veins.

### Example 15. Formulation of a Topical Composition Comprising Imatinib (Prophetic)

A formulation can be made using imatinib free base as an active ingredient. The concentrations of the ingredients in the formulation would be about 0.5% w/w imatinib, about 2.6% DEGEE, about 7.5% PEG 40 HCO, about 88.4% purified water, and about 1.0% preservative.

The formulation is prepared by dispensing the required quantity of imatinib and adding it to a suitable sized container. The required quantity of DEGEE is added to the container along with a magnetic stir bar and the mixture is stirred until the solids dissolve. PEG 40 HCO is a semisolid at room temperature and has a melting point of approximately 50° C. It is heated to >50° C. and added to the imatinib/DEGEE mixture, and the three ingredients are mixed until uniform. The required quantity of purified water is added to the mixture while stirring to create a transparent nano-emulsion followed by the addition of a preservative. The resulting formulation can be used topically to treat vitiligo and melasma.

### Example 16. Formulation of a Topical Composition Comprising Imatinib Mesylate (Prophetic)

A formulation can be made using imatinib mesylate as an active ingredient. The concentrations of the ingredients in the formulation would be about 0.5% w/w imatinib mesylate, about 2.6% DEGEE, about 7.5% PEG 40 HCO, about 88.4% purified water, and about 1.0% preservative.

The formulation is prepared by dispensing the required quantity of imatinib mesylate and adding it to a suitable sized container. The required quantity of DEGEE is added to the container along with a magnetic stir bar and the mixture is stirred until the solids dissolve. PEG 40 HCO is a semisolid at room temperature and has a melting point of approximately 50° C. It is heated to >50° C. and added to the imatinib mesylate/DEGEE mixture, and the three ingredients are mixed until uniform. The required quantity of purified water is added to the mixture while stirring to create a transparent nano-emulsion followed by the addition of an preservative. The resulting formulation can be used topically to treat vitiligo and melasma.

### Example 17. Formulation of a Topical Composition Comprising Rutin, Trans-Resveratrol, and Diosmin (Prophetic)

A formulation can be made using rutin, trans-resveratrol, and diosmin as active ingredients. The concentrations of the ingredients in the formulation would be about 0.05% w/w rutin, about 0.15% resveratrol, about 2.6% DEGEE, about 7.5% PEG 40 HCO, 1.0% preservative, about 1.0% xanthan gum, and about 87.75% purified water.

The formulation is prepared by dispensing the required quantity of rutin, trans-resveratrol, and diosmin, and adding the three ingredients to a suitable sized container. The required quantity of DEGEE is added to the container along with a magnetic stir bar and the mixture is stirred until the solids dissolved. PEG 40 HCO is a semisolid at room temperature and has a melting point of approximately 50° C. It is heated to >50° C. and added to the rutin/trans-resveratrol/diosmin/DEGEE mixture, and the ingredients are mixed until uniform. The required quantity of purified water is added to the mixture while stirring to create a transparent nano-emulsion followed by the addition of a preservative and xanthan gum. The resulting formulation can be used topically to treat varicose veins.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that variations and modifications may be made while remaining within the spirit and scope of the invention.

What is claimed is:

1. A substantially or completely oil-free aqueous emulsion comprising:
(i) about 2.5% to about 50% by weight of diethylene glycol monoethyl ether (DEGEE);
(ii) about 5% to about 20% by weight of a polyethylene glycol (PEG: H(OCH₂CH₂)ₙOH) derivative of castor oil, where n is 2 to about 1,000;
(iii) about 60% to about 92% by weight of water; and
(iv) about 0.01% to about 2% by weight of a poorly water-soluble chemical compound dispersed in the emulsion;
wherein the aqueous emulsion is substantially or completely devoid of an oil phase.

2. The aqueous emulsion of claim 1, wherein the chemical compound comprises a natural product.

3. The aqueous emulsion of claim 1, wherein the chemical compound comprises fisetin, trans-resveratrol, a milk thistle extract comprising silymarin, quercetin, dihydroquercetin, curcumin, turmeric, (−)-epigallocatechin-3-gallate (EGCG), rutin, salicylic acid, diosmin, imatinib, imatinib mesylate, and/or an extract of Centipeda minima.

4. The aqueous emulsion of claim 3, wherein the chemical compound comprises:
(i) fisetin, trans-resveratrol, and/or a milk thistle extract comprising silymarin;
(ii) EGCG and/or quercetin or dihydroquercetin;
(iii) salicylic acid; or
(iii) rutin, trans-resveratrol, and/or diosmin.

5. The aqueous emulsion of claim 1, wherein the PEG derivative of castor oil comprises PEG 40 hydrogenated castor oil (PEG 40 HCO).

6. The aqueous emulsion of claim 1, comprising:
(i) about 2.5% to about 20% by weight of DEGEE; and
(ii) about 5% to about 10% by weight of the PEG derivative of castor oil.

7. The aqueous emulsion of claim 1, wherein the chemical compound comprises fisetin, trans-resveratrol, and/or a milk thistle extract comprising silymarin, in the following proportions:
(i) about 0.075% to about 0.25% by weight of fisetin;
(ii) about 0.075% to about 0.15% by weight of trans-resveratrol; and/or
(iii) about 0.125% to about 0.25% by weight of the milk thistle extract comprising about 0.1% to about 0.2% by weight of silymarin.

8. The aqueous emulsion of claim 1, wherein the chemical compound comprises about 0.05% to about 0.1% of quercetin or dihydroquercetin.

9. The aqueous emulsion of claim 1, wherein the chemical compound comprises:
(i) about 0.03% to about 0.1% of curcumin or turmeric; and/or
(ii) about 1% to about 2% of (−)-epigallocatechin-3-gallate (EGCG).

10. The aqueous emulsion of claim 1, wherein the chemical compound comprises about 0.05% to about 0.1% of quercetin or dihydroquercetin.

11. The aqueous emulsion of claim 1, wherein the chemical compound comprises about 0.1% to about 1% of salicylic acid.

12. The aqueous emulsion of claim 1, wherein the chemical compound comprises:
(i) about 0.25% to about 0.75% of rutin; and/or
(ii) about 0.075% to about 0.15% by weight of trans-resveratrol.

13. The aqueous emulsion of claim 1, comprising nano-particles having a d50 particle size and a number weighted mean particle size each of less than about 150 nm.

14. The aqueous emulsion of claim 1, wherein the aqueous formulation is formulated to be administered to a human or animal in need of the aqueous emulsion topically, ocularly, mucosally, intravenously, intramuscularly, or orally.

15. The aqueous emulsion of claim 14, wherein the aqueous emulsion is formulated for topical administration.

16. A method to promote hair growth, or treat a skin disorder or a varicose vein, in a human or animal in need thereof, the method comprising topically applying a therapeutically effective amount of a substantially or completely oil-free aqueous emulsion for a therapeutically effective time to the skin of the human or animal, wherein the aqueous emulsion comprises:
(i) about 2.5% to about 50% by weight of diethylene glycol monoethyl ether (DEGEE);
(ii) about 5% to about 20% by weight of a polyethylene glycol (PEG: H(OCH₂CH₂)ₙOH) derivative of castor oil, where n is 2 to about 1,000;
(iii) about 60% to about 92% by weight of water; and
(iv) about 0.01% to about 2% by weight of a poorly water-soluble chemical compound dispersed in the emulsion; and
wherein the aqueous emulsion is substantially or completely devoid of an oil phase.

17. The method of claim 16, wherein the chemical compound comprises fisetin, trans-resveratrol, a milk thistle extract comprising silymarin, quercetin, dihydroquercetin, curcumin, turmeric, (−)-epigallocatechin-3-gallate (EGCG), rutin, salicylic acid, diosmin, imatinib, imatinib mesylate, and/or an extract of Centipeda minima.

18. The emulsion of claim 17, wherein the chemical compound comprises:
(i) fisetin, trans-resveratrol, and/or a milk thistle extract comprising silymarin;
(ii) EGCG and/or quercetin or dihydroquercetin;
(iii) salicylic acid; or
(iii) rutin, trans-resveratrol and/or diosmin.

19. A method of making a substantially or completely oil-free aqueous emulsion, the steps comprising:
(i) dissolving about 0.05% to about 2% by weight of a poorly water-soluble chemical compound in about 2.5% to about 50% by weight of diethylene glycol monoethyl ether (DEGEE);
(ii) diluting the mixture of step (i) with about 60% to about 92% by weight of water; and
(iii) adding about 5% to about 20% by weight of a polyethylene glycol (PEG: H(OCH₂CH₂)ₙOH) derivative of castor oil to the mixture of step (ii), where n is 2 to about 1,000;
wherein the aqueous emulsion is substantially or completely devoid of an oil phase.

20. The method of claim 19, wherein the chemical compound comprises fisetin, trans-resveratrol, a milk thistle extract comprising silymarin, quercetin, dihydroquercetin, curcumin, turmeric, (−)-epigallocatechin-3-gallate (EGCG), rutin, salicylic acid, diosmin, imatinib, imatinib mesylate, and/or an extract of Centipeda minima.
